(19) United States
(12) Patent Application Publication
(10) Pub. No.: US 2011/0064676 A1
(43) Pub. Date: Mar. 17, 2011

(54) Diagnostic and therapeutic nanoparticles

(75) Inventors: Andre' M. Gobin, Shepherdsville, KY (US); Guandong Zhang, Louisville, KY (US)

(73) Assignee: University of Louisville Research Foundation, Inc.

(21) Appl. No.: 12/807,792

(22) Filed: Sep. 14, 2010

Related U.S. Application Data
(60) Provisional application No. 61/276,850, filed on Sep. 17, 2009.

(51) Int. Cl. A61K 49/04; A61K 9/14; A61K 49/00; A61K 39/395; A61P 35/00

(52) U.S. Cl. 424/9.42; 424/490; 424/493; 424/9.1; 424/130.1; 424/9.4; 977/773; 977/810; 977/906; 977/927; 977/928

(57) ABSTRACT

The present invention relates to diagnostic and therapeutic nanoparticles. More particularly, the present invention relates to creating a hybrid gold/gold sulfide nanoparticle with a chitosan matrix surrounding the metallic nanoparticle and a method for making the same. The chitosan-coated gold/gold sulfide nanoparticles can then be incorporated with additional therapeutic or diagnostic compounds such as iodine, antibodies, or other suitable compounds. The nanoparticles of the present invention have the dual capabilities of absorbing near infrared wavelength light to (1) act as a therapeutic agent by generating heat energy effective for cell ablation or for release of therapeutic compounds embedded in the chitosan matrix and (2) creating diagnostic benefit by incorporation of X-ray or MRI contrast-agents.

This application claims the benefit of U.S. Provisional Patent Application Ser. No. 61/276,850, entitled DIAGNOSTIC AND THERAPEUTIC NANOPARTICLES, to André M. Gobin and Guandong Zhang, filed Sep. 17, 2009 and incorporated herein by reference.

### BACKGROUND OF THE INVENTION

(a) Field of the Invention

The present invention relates to diagnostic and therapeutic nanoparticles. More particularly, the present invention relates to creating a hybrid gold/gold sulfide nanoparticle with a chitosan matrix surrounding the metallic nanoparticle and a method for making the same. The chitosan-coated gold/gold sulfide nanoparticles can then be incorporated with additional therapeutic or diagnostic compounds such as iodine, antibodies, or other suitable compounds. The nanoparticles of the present invention have the dual capabilities of absorbing near infrared wavelength light to (1) act as a therapeutic agent by generating heat energy effective for cell ablation or for release of therapeutic compounds embedded in the chitosan matrix and (2) creating diagnostic benefit by incorporation of X-ray or MRI contrast agents.

(b) Description of the Prior Art

Hepatocellular carcinoma (“HCC”) affects greater than half a million patients worldwide. U.S. liver-related cancer deaths account for 4% of all cancers or about 20,000 deaths annually. Currently available minimally invasive procedures have the tendency to leave a population of malignant cells intact, allowing for recurrence of the tumor and accounts for the varied recurrence rates seen. This recurrence is equivalent across all therapeutic modalities, including radiofrequency ablation, laser induced thermotherapy and transarterial chemoembolization, with approximately 20% residual viability at time of liver explantation during liver transplantation.

The incident rate of prostate cancer in men was 33% of all cancers in the U.S. in 2004. This incident rate is similar to the 31% incident rate of breast cancer in women in the United States. These rates stay relatively unchanged through 2006. The number of deaths in 2006 due to prostate cancer in the U.S. was 26,214, or 9% of all cancer related deaths in men. Prostate cancer easily metastasizes, increasing the chance of death if not caught early. In the current stage of detection through prostate specific antigen (PSA), at least 25% of U.S. men tested as present with metastases to the bone. These men have a 90% risk of death within five years. The high mortality rates of these cancers after metastasis is a significant health risk.

Nanoparticles can be efficiently delivered into cancerous tissue, such as tumors, via a property inherent of fast growing neoplasias called Enhanced Permeability and Retention (“EPR”). This property is marked by “leaky vasculature” within tumors, allowing extravasation and retention of macromolecules or nanoscale particles. Silica core/gold nanoshells (composite nanoparticles) which have tunable plasmon resonance in the near infrared (“NIR”) have been used to induce sufficient thermal damage after accumulation via this mechanism leading to tumor reduction and increased survival. Using light based imaging techniques (optical coherence tomography or OCT) these nanoparticles have been used as a theranostic (i.e., combined diagnostic and therapeutic) agent in a single nanoshell formulation. Though these particles may provide effective diagnostic capabilities for shallow (<5 mm) tumors they are unsuitable as a good diagnostic approach where tumors may be deeply situated.

Pure gold nanoparticles or colloidal gold has been shown to produce very high contrast increase with X-ray techniques in animal studies, allowing detailed visualization of blood vessels and soft tissue structures not possible before, and thus offering the possibility of detecting tumors deeply seated in soft tissues. These particles show great promise as a CT contrast agent but have several inherent shortcomings. The first shortcoming is that dosages of colloidal gold used to effect CT contrast is up to 200 times greater than the dosages of NIR absorbing gold nanoshells needed to effect tumor reduction and remission. The second shortcoming is that colloidal gold has no therapeutic value. Finally, even with anti-fouling coatings, the number of particles being used for treatment could potentially overwhelm the reticuloendothelial system (“RES”), thus it is always necessary to minimize the number of particles injected.

Heavy elements like iodine can provide excellent contrast for X-ray imaging but are often associated with cytotoxic effects in the kidney (nephrotoxicity). Thus other iodinated compounds and liposomal encapsulated iodine compounds have been formulated and are currently available as contrast agents; these are widely used in HCC imaging using multi-detector computed tomography (MDCT) with excellent imaging results. Many of these iodinated compounds embedded in polymeric nanoparticles show excellent contrast increase and minimal toxicity in vitro.

A need exists for single theranostic agent with the therapeutic tunable plasmon resonance in the NIR capability of gold nanoshells and the increased X-ray and. CT contrast offered by pure gold nanoparticles and iodine. Use of a single agent minimizes procedures by removing the need for two separate procedures for diagnosis and treatment. Combining the capabilities allows faster treatment of patients when tumors are detected and can save lives and improve the reach of treatment to a wider population.

### SUMMARY OF THE INVENTION

The aforementioned problems have been solved in the present invention. To effectively combine the therapeutic benefit of NIR absorbing gold/silica nanoshells with the contrast increase of pure gold and that of iodinated compounds, the present invention provides a hybrid nanoparticle of NIR absorbing gold/gold sulfide nanoparticles within an iodine-containing chitosan matrix. In addition to HCC treatment, this technology has the potential to impact many of the greater than 500,000 annual deaths from cancer, including prostate cancer and most other major forms of cancer, which together combine to be the second leading cause of death in the United States. As these hybrid nanoparticles are smaller than silica core/gold nanoshells, the invention will allow diagnostic and treatment of smaller tumors, including those arising from metastatic events from prostate and liver cancers. Further, this hybrid nanoparticle could potentially be used in non-cancer imaging applications including coronary and cerebral arteries, atherosclerotic plaques and stenoses as well as in mammography where fast 3D visualization afforded by MDCT will enhance diagnosis of these conditions.

The present invention provides a novel hybrid nanoparticle with enhanced opacity to X-rays for unprecedented imaging contrast while retaining the ability to be activated by NIR light for therapeutic purposes. The present invention also includes manufacturing techniques for producing large quantities of these NIR absorbing gold/gold sulfide nanoparticles with strong absorption of NIR energy (>98% efficiency). The composite structure of these nanoparticles is closer in density (18.5 g/cc) to that of pure gold nanoparticles (19.3 g/cc) than gold/silica nanoshells (8.4 g/cc), thus allowing the hybrid nanoparticles to act like the dense pure gold nanoparticles in terms of their ability to block X-rays. In addition, techniques for effectively coating these hybrid nanoparticles with the bioderived polymer chitosan have been developed and show extreme stability in physiologically relevant ionic ranges. Further, by combining the gold/gold sulfide nanoparticles with iodine via the chitosan matrix on its surface, the X-ray opacity of the nanoparticle will be enhanced. Use of iodine in a bound nanoparticle reduces the free iodine in circulation and reduces potential nephrotoxicity. The complexation of iodine with chitosan has been previously demonstrated to form an irreversible bond with thermal hysteresis (showing stability). The NIR properties of the gold/gold sulfide nanoparticles remain and the new hybrid will be capable of performing as a contrast agent for CT as well as a therapeutic agent. Further, chemical conjugation of existing molecules used for CT contrast can be achieved with the chitosan matrix, allowing extension of the system to enhance currently available contrast agents.

Nanoshells are a relatively new class of engineered nanoparticles consisting of an ultrathin metal shell surrounding a dielectric core. Gold coated nanoshells have properties making them ideal for biological applications, including good biocompatibility, and tunable optical properties. Nanoshells can be designed to either absorb strongly or scatter light in the NIR based on the total size and the ratio of radii of the core and shell, permitting applications for heating or optical contrast. Gold nanoshells have been investigated for a variety of biomedical applications, including use as a mechanism to provide heating for photo-thermally modulated drug delivery systems, a fast antigen detection systems with whole blood, and for use in imaging applications. Nanoshells have also been investigated for use as an NIR absorber for cancer therapy by non-specific accumulation in tumors and for targeted cell ablation.

Gold/silica (Au/SiO₂) nanoshells are generally produced with a diameter of 120-150 nm for current medical applications, though particles with diameters as large as 400 nm have been produced. These nanoshells can be made with silica cores as small as 100-120 nm with shell thickness ranging between about 10-15 nm. At these sizes, approximately 67-85% of the incident energy is converted to heat and the balance is scattered (larger particles scatter more light), thus allowing imaging or detection via light. Tunability is important as it allows creating of nanoparticles with optical absorption in the near infrared (NIR) region: wavelengths between 700-900 nm. In this range, there is minimal absorption of energy by tissue components of water and hemoglobin. By coupling NIR laser energy with innocuous exogenous chromophores, such as nanoshells, one can enable photothermal therapies. Particles in this size range have been used for treatment in murine carcinoma models of fast growing tumors showing up to 100% regression. These tumor models, subcutaneous murine colon carcinoma, have the leaky vasculature associated with fast growing tumors allowing use of macromolecules and nanotherapeutic agents.

The present invention relates to smaller, denser nanoparticles comprising of a composite of gold and gold sulfide, which can also be produced to have strong NIR absorption. These gold/gold sulfide (“GGS”) nanoparticles are formed by self assembly of gold and sulfide. These nanoparticles are distinct from nanoshells as they do not include a discrete core section surrounded by a shell layer. Furthermore, GGS nanoparticles self assemble in solution such that there is no need to deposit seed molecules or use linker molecules to assemble the nanoparticles. GGS nanoparticles are also significantly smaller and more dense than gold nanoshells, and include an, intrinsic CT and X-ray contrast functionality not shared by gold nanoshells.

During self-assembly of GGS nanoparticles, gold colloid is simultaneously formed. These gold colloids are similar to those discussed for CT contrast by others and are smaller that the GGS nanoparticles and have absorption of about 530 nm. These colloids are considered contaminants of the therapeutic nanoparticle production process, but are easily removed through centrifugation, rendering the NIR particles available for use in biomedical applications.

GGS nanoparticles with 800-860 nm peak resonance are on the order of 35-55 nm in diameter and simulation shows that these small particles absorb and efficiently convert up to 99% of the incident energy. GGS nanoparticles with 800-840 nm peak resonance are on the order of 35-45 nm in diameter and also absorb and efficiently convert incident energy. The size of the particle determines its precise peak resonance. These smaller, more efficiently absorbing GGS nanoparticles provide easier access to tumors than the larger gold/silica nanoshells. GGS nanoparticles access tumors for NIR photothermal therapy by extravasating in tumors with smaller fenestrations in the vasculature and by allowing heating of the nanoparticles at either a) greater depth or b) lower light energy due to the efficiency by which they convert light to heat.

Although the ability to target a particle or drug to a specific cell line in vitro has become fairly straightforward and widespread using a variety of'approaches, localization of the agent out of circulation and into the tumor is still mainly dependent on the EPR effect. Targeting allows the agent to be better taken up by the malignant cell line once the particle is within the tumor. The present invention uses a naturally derived polymer, chitosan, to act as the mechanism for increased uptake to the target cell line. Use of chitosan nanoparticles have shown effective targeting in vitro and uptake directly to HCC cells in vivo providing tumor reduction.

Chitosan is deactylated chitin, the structural component of shrimp and crab shells. Deacetylation leaves a free amine group that becomes protonated and imparts a positive charge to the molecule. Chitosan is a cationic polysaccharide of D-glucosamine and N-acetyl-D-glucosamine. It has several characteristics such as biocompatibility, biodegradability, positive charge, nontoxicity, bioadhesivity, and antibiotic properties which make this macromolecule useful for many biological application. In particular, the bioadhesivity of chitosan is based on electrostatic interactions and does not require mediation by cellular components. Chitosan has been developed into nanoparticles as drug delivery vehicles, complexed with DNA for non-viral transfection of cells and used in tissue engineering. The positive charge of chitosan is used in this invention to self assemble chitosan onto negatively charged GGS nanoparticles, yielding hybrid nanoparticles. The diameter of a hybrid nanoparticle, i.e., the GGS nanoparticle and its surrounding chitosan matrix when measured by tunneling electron microscopy (“TEM”) in a dried state, is between 45 nm and 100 nm in diameter, preferably between 45 nm and 75 nm in diameter, or ideally about 50 nm in diameter. All dimensions discussed in connection with the chitosan matrix refer to the present invention in a dried state. While the size of the GGS nanoparticle remains constant whether dried or in an aqueous environment, the hydrodynamic size of the hybrid nanoparticle as a whole can be significantly larger than the dried size due to expansion of the chitosan matrix.

The function of chitosan in the present invention is threefold. First, it acts as a matrix to allow binding of agents, such as iodine for imaging contrast increase and/or therapeutic or diagnostic agents, such an antibodies or pharmaceuticals for targeted drug delivery. Second, chitosan has been shown to cause tumor regression in HCC when formulated as a nanoparticle. Third, chitosan increases the biocompatability of the hybrid nanoparticle. While chitosan may be strongly positive at certain pH levels, it is not a conducting material.

In one embodiment, thiolated chitosan is used to increase the binding strength to the surface of the nanoparticle. In another embodiment, carboxymethylated chitosan (“CMCS”) may be used. GGS nanoparticles with chitosan and CMCS coatings have isoelectric points of about 7.7 and 6.1, respectively. By assembling chitosan, CMCS, or a blend thereof upon a GGS nanoparticle, the isoelectric point of the resulting hybrid nanoparticle can be tuned. This is an important feature of the present invention which allows the hybrid nanoparticle to be customized for use in biological systems with different acidities, such as the stomach and blood stream.

Iodine, chemical element number 53, has been used is a variety of medical applications for decades, including as an antiseptic in alcohol, radio-protective measure for radiation exposure, treatment for thyroid cancer and as a CT contrast agent due to its ability to block X-rays. In this invention, iodine is a key element in allowing the increase of contrast in the hybrid nanoparticle system. Iodine, being a halogen, forms a negatively charged ion when its salts are dissolved. Iodine from potassium iodide or other compounds can be used in the self assembly technique to incorporate into the positively charged chitosan matrix. At therapeutic concentrations of nanoparticles, on the order of 10¹⁰-10¹¹ particles, CT contrast will be enhanced with the use of iodine.

In one embodiment, the GGS nanoparticle may also include gadolinium to further enhance imaging contrast via MRI. Incorporation into the hybrid nanoparticle can be accomplished by using the electrostatic attraction of the positively charged gadolinium to the surface of the nanoparticle which is stabilized by the chitosan matrix.

Polyethylene glycol (“PEG”) is a very hydrophilic polymer at higher molecular weights (>1000 g/mol). As it does not interact with proteins, it is used in a variety of applications where lack of protein adhesion is required, essentially forming an anti-fouling surface. As such, it has been used to reduce opsonization of many nanoparticles including gold nanoparticles. This allows the nanoparticle to become somewhat invisible to the body's immune system, avoiding uptake by the RES, thus increasing circulation of macromolecules and nanoparticles. Chitosan nanoparticles have been used directly in vivo without PEG modification; however, PEG may be used in this invention as the final addition to the hybrid nanoparticle to reduce removal by the RES.

The theranostic nanoparticle of the present invention will benefit patients with many forms of cancer. The benefits will include better ability to diagnose primary as well as metastatic cancer events due to the hybrid nanoparticle's small size, less than about 75 nm, thus allowing accumulation within even small, tumors. Once located via CT scan, these nanoparticles can be activated by the use of directed NIR light to optically heat the nanoparticles through placement of diffuse fiber optics or through the use of radio frequency ablation currently being used in HCC treatment. This will allow heating of the nanoparticles for immediate killing of cancerous cells containing the nanoparticles, where the heating causes an increase in temperature of 12-18° C. above body temperature. As EPR causes nanoparticles to accumulate in tumor cells, normal cells will remain unharmed. Studies by the inventors indicate that the hybrid nanoparticles disclosed herein may be particularly effective at treating esophageal carcinomas without damaging healthy tissue.

In addition to its uses in directed hyperthermia and as a CT contrast agent, the theranostic nanoparticles disclosed herein may also act as a drug delivery system by including embedded agents. Heating the nanoparticles will cause release of agents embedded in the nanoparticle's chitosan matrix for drug release and continued action after energy source is removed. The releasably embedded agents may be diagnostic agents or therapeutic agents, such as, for example, any desired pharmaceutical or antibody. An important aspect of the present invention is that both a diagnostic step (the CT scan) and a therapeutic step (the ablation and/or agent release) may be accomplished using a single compound (the hybrid nanoparticles).

Novel manufacturing techniques for large scale production (>1 liter/batch) of the theranostic agent of the present invention will make GGS nanoparticles widely available and at comparatively low cost. The use of self-assembly techniques to build the hybrid nanoparticle reduces costs associated with manufacturing throughout the process. The use of chitosan as the encapsulating matrix around the gold nanoparticle will allow the use of cheaper and more widely available materials into the hybrid nanoparticle, as well as removing the expense associated with using aptamers or antibodies to bind HCC cells. Finally, the use of iodine as a contrast agent reduces costs compared to increasing the contrast by increasing the gold concentration. These methods will improve the reach of a cancer diagnostic and therapeutic agent providing greater impact and reduced health-care costs. By using our approach to producing large quantities of GGS nanoparticles the cost of the theranostic agent will be reduced as the technology comes to market.

In one embodiment, the present invention is a process for making a hybrid nanoparticle with tunable resonance peak comprising the steps of: (a.) providing a gold source; (b.) providing a sulfide source; (c.) combining the gold source and the sulfide source for self-assembly of a nanoparticle comprising gold and gold sulfide; and (d.) coating the nanoparticle with a bioderived coating; wherein the resonance peak of the nanoparticle may be tunable by adjusting the ratio of gold source and sulfide source used in step (c.). In this embodiment, the bioderived coating may be a chitosan coating, which may include modified chitosan, such as carboxymethylated chitosan or triiodobenzoic acid-modified chitosan. In this embodiment, the biodervied coating may be a mixture of biodervied coatings which may include chitosan, such as a mixture of chitosan and carboxymethlyated chitosan. In this embodiment, the bioderived coating may self-assemble onto the nanoparticle. This embodiment may further comprise step (e.): incorporating at least one of a therapeutic agent, a diagnostic agent, and a contrast agent into the hybrid nanoparticle. The at least one of a therapeutic agent, a diagnostic agent, and a contrast agent may be embedded in the bioderived coating. The at least one of a therapeutic agent, a diagnostic agent, and a contrast agent may be an antibody, or a pharmaceutical. In this embodiment, the sulfide source may be sodium thiosulfate and the gold source may be cholorauric acid.

In a further embodiment, the present invention is a method for tuning the isoelectric point of a nanoparticle comprising the step of coating the nanoparticle with a bioderived coating comprising a mixture of chitosan and carboxymethylated chitosan, whereby the isoelectric point may be tuned by varying the ratio of chitosan and carboxymethylated chitosan.

In another embodiment, the present invention is a method for using theranostic hybrid nanoparticles comprising the steps of: (a.) providing optically heatable hybrid nanoparticles having diameters less than about 100 nm and improved contrast functionality with X-ray and CT imaging; (b.) delivering the hybrid nanoparticles to a specific tissue; (c.) imaging the tissue using at least one of X-ray and CT imaging; (d.) optically heating the hybrid nanoparticles located at the specific tissue, whereby optically heating the hybrid nanoparticles results in at least one of: (1.) thermal ablation of at least a portion of the specific tissue; (2.) release of a diagnostic agent incorporated within the hybrid nanoparticles; (3.) release of a therapeutic agent incorporated within the hybrid nanoparticles. In this embodiment, each of the hybrid nanoparticles may be comprised of a nanoparticle comprising gold and gold sulfide, and a bioderived coating assembled on the nanoparticle. In this embodiment, a contrast agent may be incorporated within the bioderived coating and the contrast agent may not be released upon optically heating the hybrid nanoparticle. In this embodiment, the specific tissue may be a cancerous tissue.

In a further embodiment, the present invention is a method for delivering a therapeutic or diagnostic agent to a specific tissue comprising the steps of: (a.) providing optically heatable hybrid nanoparticles comprised of: (1.) a nanoparticle comprising gold and gold sulfide; (2.) a bioderived coating assembled on the nanoparticle; (3.) at least one agent from the group consisting of a therapeutic agent and a diagnostic agent, the agent releaseably incorporated within the bioderived coating; (b.) delivering the hybrid nanoparticles to a specific tissue; (c.) optically heating the hybrid nanoparticles located at the specific tissue, whereby optically heating the hybrid nanoparticles results in release of the at least one agent.

### BRIEF DESCRIPTION OF THE DRAWINGS

A better understanding of the present invention will be had upon reference to the following description in conjunction with the accompanying drawings, wherein:

FIG. 1 is a graph of the NIR peak wavelength of a GGS nanoparticle as a function of its molar ratio of HAuCl₄/Na₂S₂O₃; and

FIG. 2 is a graph of the diameter of a GGS nanoparticle as a function of its absorbance peak wavelength.

### DETAILED DESCRIPTION OF THE PREFERRED EMBODIMENT

The theranostic hybrid nanoparticle of the present invention comprises a GGS nanoparticle and a matrix of chitosan self-assembled on the charged GGS nanoparticle surface. In some embodiments, the hybrid nanoparticle further include a CT contrast agent, such as iodine, and/or a therapeutic agent or diagnostic agent. The term hybrid nanoparticle is used herein to collectively refer to a GGS nanoparticle and any coating, agent, or other material attached thereto.

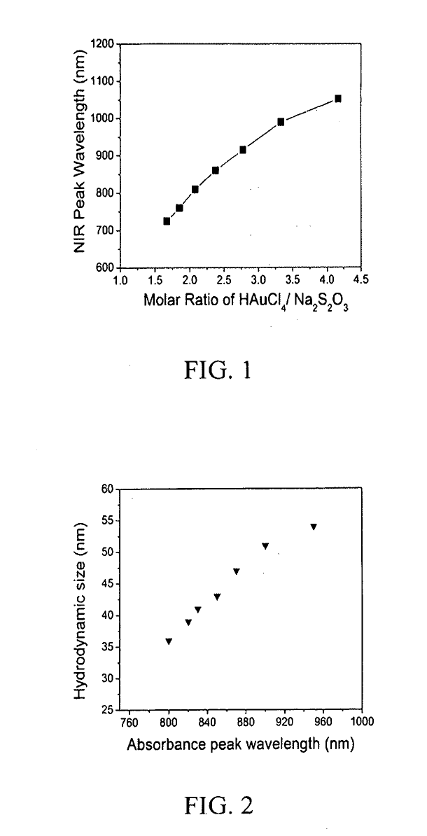

GGS nanoparticles are preferably created by the reaction of a sulfide source, sodium thiosulfate, and a gold source, cholorauric acid. The reaction between Na₂S₂O₃ (sodium thiosulfate) and HAuCl₄ (chloroauric acid) is easier to control compared to the reaction between Na₂S and HAuCl₄. The resonance peak of GGS nanoparticles can be controlled by cooperatively adjusting the ratio of sodium thiolsulfate and chloroauric acid solutions used to create the GGS nanoparticles. In addition, the concentration of each component may also effect the resonance peak. As shown in FIG. 1, at a set concentration of each component, the wavelength of the absorbance peak increases as the proportion of sodium thiosulfate decreases. Using manufacturing methods disclosed herein, GGS nanoparticles may be produced with resonances between about 600 nm and about 1100 nm.

As shown in FIG. 2, the peak optical absorption of a GGS nanoparticle is related to its diameter, which in turn is related to the ratio of chloroauric acid to sodium thiosulfate used to create the GGS nanoparticle. In a preferred embodiment, the GGS nanoparticles are about 35-55 nm in diameter, which result in an absorbance peak in the range of about 800-860 nm. In an ideal embodiment, the GGS nanoparticles are about 35-45 nm in diameter, which results in an absorbance peak of about 800-840 nm. Absorption in these wavelengths is ideal for GGS nanoparticles as there is minimal absorption of energy by tissue components of water and hemoglobin. The addition of chitosan to the GGS nanoparticle increases the absorbance peak for the hybrid nanoparticle, the increase amount varying based on the amount and composition of chitosan used.

Colloidal gold is a byproduct of reacting sodium thiosulfate and cholorauric acid. GGS nanoparticles are separated from colloidal gold by centrifugation. An example separation process is centrifugation at 1000 g for 20 minutes. Additional centrifugation steps may be used to increase yield.

Chitosan is added between 0.01 wt % and 0.10 wt % chitosan/optical density (“OD”). Preferably, the chitosan concentration is equal to or less than about 0.02 wt %/OD. At higher concentrations of chitosan, high viscosity hinders the separation of nanoparticles from solution. Chitosan forms a 1-20 nm thick layer on the surface of the GGS nanoparticle. Unmodified chitosan tends to form layers between about 1-5 nm in thickness, while CMCS and mixtures of CMCS and unmodified chitosan tend to form layers between about 10-20 nm in thickness. Chitosan adsorption requires at least about 4 hours and preferably about one day for the coated GGS nanoparticle size to stabilize. Chitosan is added to the gold/gold sulfide solution 30-60 minutes, preferably about 45 minutes, after initiating the reaction to form nanoparticles. Early addition of chitosan allows a stronger bond of the chitosan matrix to the surface of the GGS nanoparticle, thus providing a denser chitosan coating after the reaction. However, if chitosan is added too early after the reaction of chloroauric acid to sodium thiosulfate, it blocks the nanoparticle surface, inhibiting nanoparticle growth and reducing yield.

In some embodiments, iodine, from iodinated compounds such as triiodobenzoic acid and potassium iodide, may be conjugated to chitosan and added to the GGS nanoparticles. The negatively charged iodide ion will naturally incorporate into the positively charged chitosan matrix. Addition of iodine can occur in a variety of ways, including direct mixing after formation of the chitosan-coated GGS nanoparticles or by incorporation of the iodine with chitosan electro-statically or covalently prior to the addition to the reaction mixture as detailed above. In a preferred embodiment, chitosan is modified by coupling with 3,4,5-triiodobenzoic acid (“TIBA”) through the 1-ethyl-3-(3-dimethylaminopropyl)-carbodiimide (“EDC”) mediated reaction before adding the TIBA-modified chitosan to the GGS nanoparticles. The exact amount of iodine added would vary based on the intended use of the nanoparticles. TIBA-modification may be used with unmodified chitosan, CMCS, or a mixture thereof.

In addition to its uses in directed hyperthermia and as a CT contrast agent, the theranostic nanoparticles of the present invention may also include embedded agents. Heating the nanoparticles will cause release of agents embedded in the nanoparticle's chitosan matrix for drug release and continued action after energy source is removed. In one embodiment, negatively charged agents will embed within the chitosan matrix due to electrostatic interactions. In another embodiment, linker molecules may be used to covalently or, preferably, electrostatically cross-link chitosan molecules within the matrix, trapping agents within the matrix. When the nanoparticle is heated, the linker molecules release, removing the cross-linking, which releases the embedded agents. The releasably embedded agents may be diagnostic agents or therapeutic agents, such as any desired pharmaceutical or antibody.

In one embodiment, the nanoparticle further comprises PEG. In a this embodiment, the nanoparticle further comprises thiolated polyethylene glycol (“SH-PEG”) (1000-2500 g/mol). In this preferred embodiment, SH-PEG is added at concentrations between 0.0025 μM and 0.02 μM. PEG shields the strong positive charge of chitosan coated nanoparticles, making them more suitable for biological applications. The surface charge of the nanoparticle decreases with increasing concentration of PEG, so varying the PEG addition allows for effective control of the nanoparticle charge. Positively charged nanoparticles are more cell attractive, but other applications may require more neutral nanoparticles. In one embodiment, after addition of PEG, the chitosan-coated GGS nanoparticles are less than about 100 nm in diameter. In a preferred embodiment, after addition of PEG, the chitosan-coated GGS nanoparticles are less than about 75 nm in diameter.

In a preferred embodiment, the chitosan-coated GGS nanoparticles of the present invention are sterilized before use in a biological system. An example sterilization procedure is to place the nanoparticles in an autoclave for 45 minutes at 121° C. Another example sterilization procedure is to pass the nanoparticles through a 0.2 micron filter. A preferred sterilization procedure is to pass the nanoparticles through a 0.8 micron filter followed by a 0.2 micron filter.

### EXAMPLE 1

This example demonstrates the preparation of a first embodiment of the hybrid nanoparticle of the present invention. This hybrid nanoparticle includes a GGS nanoparticle with an absorbance peak at about 820 nm and a chitosan coating and has an isoelectric point of about 7.7. The procedure to prepare this embodiment of the present invention is as follows.

GGS nanoparticles are prepared by the reaction of sodium thiosulfate and chloroauric acid. 54 ml 3 mM Na₂S₂O₃ is HAuCl₄, and vortexed for about 1 minute. The solution is then left to react for about 45 minutes. The nanoparticle concentration is around 3.5 to 4 OD.

Low molecular weight (“LMW”) chitosan, such as that provided by Sigma-Aldrich, is used for the coating of GGS nanoparticles. The chitosan solution is prepared by dissolving 1.0 g LMW chitosan in 100 ml 0.7 wt. % acetic acid solution.

The chitosan is added to the GGS nanoparticle solution about 45 minutes after the mixing of chloroauric acid and sodium thiosulfate solutions. The weight ratio between chitosan and GGS nanoparticles is about 0.02 wt. % chitosan/OD. Allow at least four hours for adsorption of chitosan onto the surface of nanoparticles.

The hybrid nanoparticle solution is sterilized by passing the solution through a 0.2 micron filter.

Hybrid nanoparticles are separated from solution by three rounds of centrifugation at 1000 g for 20 minutes each round.

### EXAMPLE 2

This example demonstrates the preparation of a second embodiment of the hybrid nanoparticle of the present invention. This hybrid nanoparticle includes a GGS nanoparticle with an absorbance peak at about 820 nm and a TIBA-modified chitosan coating and has an isoelectric point of about 7.7. The procedure to prepare this embodiment of the present invention is as follows.

GGS nanoparticles are prepared by the reaction of sodium thiosulfate and chloroauric acid. 54 ml 3 mM Na₂S₂O₃ is added to 150 ml 2 mM HAuCl₄, and vortexed for about 1 minute. The solution is then left to react for about 45 minutes. The nanoparticle concentration is around 3.5 to 4 OD.

TIBA-modified chitosan is used for the coating of GGS nanoparticles. The TIBA-modified chitosan solution is prepared by dissolving 0.4 g LMW chitosan in 40 ml 0.7 wt. % acetic acid solution. The chitosan solution is then dialysed in DI water for 2 to 6 days. The pH of the chitosan solution increases from about 4.0 to about 6.0-6.3 after dialysis. 0.20 g TIBA is dissolved in a solvent containing 30 ml methanol and 10 ml tetrahydrofuran. The chitosan solution is then slowly added to the TIBA solution with smooth agitation. 10 ml of EDC (20 mg/ml) is then added drop-wise to the reaction solution. The reaction mixture is stirred at room temperature. After 24 hours, the reaction mixture is poured into 200 ml of a methanol/ammonia (7/3 v/v) solution with stirring. The precipitated material is then centrifuged at about 60 g for about 10 min, washed with a 85% methanol water solution, and dried under vacuum for 24 hours at 40° C. Before use, the dried TIBA-modified chitosan is put into solution of 0.7% acetic acid at a concentration of 1% wt TIBA-modified chitosan.

The TIBA-modified chitosan is added to the GGS nanoparticle solution about 45 minutes after the mixing of chloroauric acid and sodium thiosulfate solutions. The weight ratio between TIBA-modified chitosan and GGS nanoparticles is about 0.02 wt. % chitosan/OD. Allow at least four hours for adsorption of chitosan onto the surface of nanoparticles.

The hybrid nanoparticle solution is sterilized by passing the solution through a 0.8 micro filter followed by a 0.2 micron filter.

Hybrid nanoparticles are separated by centrifugation at 1000 g for 20 minutes.

### EXAMPLE 3

This example demonstrates the preparation of a third embodiment of the hybrid nanoparticle of the present invention. This hybrid nanoparticle includes a GGS nanoparticle with an absorbance peak at about 850 nm and a blended chitosan/CMCS coating and has an isoelectric point of about 7.1. The procedure to prepare this embodiment of the present invention is as follows.

GGS nanoparticles are prepared by the reaction of sodium thiosulfate and chloroauric acid. 28.5 ml 3 mM Na₂S₂O₃ is added to 150 ml 2 mM HAuCl₄, and vortexed for about 1 minute. The solution is then left to react for about 45 minutes. The nanoparticle concentration is around 3.5 to 4 OD.

A blend of LMW chitosan and CMCS is used for the coating of GGS nanoparticles. The chitosan solution is prepared by dissolving 1.0 g LMW chitosan in 100 ml 0.7 wt. % acetic acid solution. CMCS is prepared by dissolving 15 g sodium hydroxide in a mixture solution of 80 ml isopropanol and 20 ml DI water. 10 g LMW chitosan is added and allowed to alkalize at 50° C. for 1 hour. 15 g monochloroacetic acid is dissolved in 20 ml isopropanol, and added to the reaction mixture dropwise over 30 minutes and allowed to react for 4 hours at 50-55° C. The reaction is then stopped by adding 200 ml 70% ethanol. The solid product is rinsed with 80% ethyl alcohol to desalt and dewater the CMCS until the pH value of the CMCS solution is less than 8.0. The CMCS is then vacuum dried at 40° C. for 1 day. Before use, the dried CMCS is put into aqueous solution at a concentration of 1% wt CMCS.

Chitosan and CMCS are added in a 3:1 ratio to the GGS nanoparticle solution about 45 minutes after the mixing of chloroauric acid and sodium thiosulfate solutions. The weight ratio between blended chitosan and GGS nanoparticles is about 0.02 wt. % chitosan/OD. Allow at least four hours for adsorption of chitosan onto the surface of GGS nanoparticles.

The hybrid nanoparticle solution is sterilized by passing-the solution through a 0.2 micron filter.

Hybrid nanoparticles are separated by centrifugation at 1000 g for 20 minutes.

EXAMPLE 4

This example demonstrates the preparation of a fourth embodiment of the hybrid nanoparticle of the present invention. This hybrid nanoparticle includes a GGS nanoparticle with an absorbance peak at about 820 nm and a chitosan coating and has an isoelectric point of about 7.7. As discussed in paragraph [0034], the addition of a chitosan coating results in an increase in the absorbance peak wavelength. In this exemplary embodiment, the absorbance peak of the hybrid nanoparticle as a whole is at about 927 nm. The procedure to prepare this embodiment of the present invention is as follows.

GGS nanoparticles are prepared by the reaction of sodium thiosulfate and chloroauric acid. 54 ml 3 mM Na₂S₂O₃ is added to 150 ml 2 mM HAuCl₄, and vortexed for about 1 minute. The solution is then left to react for about 45 minutes. The nanoparticle concentration is around 3.5 to 4 OD.

LMW chitosan, such as that provided by Sigma-Aldrich, is used for the coating of GGS nanoparticles. The chitosan solution is prepared by dissolving 1.0 g LMW chitosan in 100 ml 0.7 wt. % acetic acid solution.

16 ml 1.0 wt % LMW chitosan is added to the GGS nanoparticle solution 35-40 minutes after the mixing of chloroauric acid and sodium thiosulfate solutions. Allow at least four hours for adsorption of chitosan onto the surface of nanoparticles.

The hybrid nanoparticle solution is sterilized by passing the solution through a 0.2 micron filter.

Hybrid nanoparticles are separated from solution by three rounds of centrifugation at 1000 g for 20 minutes each round.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1. A process for making a hybrid nanoparticle with tunable resonance peak comprising the steps of:
a. providing a gold source;
b. providing a sulfide source;
c. combining said gold source and said sulfide source for self-assembly of a nanoparticle comprising gold and gold sulfide; and
d. coating said nanoparticle with a bioderived coating;
wherein the resonance peak of said nanoparticle is tunable by adjusting the ratio of gold source and sulfide source used in step c.

2. The process of claim 1, wherein said bioderived coating is a chitosan coating.

3. The process of claim 2, wherein said chitosan is modified chitosan.

4. The process of claim 3, wherein said modified chitosan is carboxymethylated chitosan.

5. The process of claim 3, wherein said modified chitosan is triiodobenzoic acid-modified chitosan.

6. The process of claim 1, wherein said biodervied coating is a mixture of biodervied coatings.

7. The process of claim 6, wherein said mixture of bioderived coatings includes chitosan.

8. The process of claim 7, wherein said mixture of bioderived coatings is a mixture of chitosan and carboxymethlyated chitosan.

9. The process of claim 1, wherein said bioderived coating self-assembles onto said nanoparticle.

10. The process of claim 1, further comprising step e:
incorporating at least one of a therapeutic agent, a diagnostic agent, and a contrast agent into said hybrid nanoparticle.

11. The process of claim 10, wherein said at least one of a therapeutic agent, a diagnostic agent, and a contrast agent is embedded in said bioderived coating.

12. The process of claim 10, wherein said at least one of a therapeutic agent, a diagnostic agent, and a contrast agent is an antibody.

13. The process of claim 10, wherein said at least one of a therapeutic agent, a diagnostic agent, and a contrast agent is a pharmaceutical.

14. The process of claim 1, wherein said sulfide source is sodium thiosulfate and said gold source is cholorauric acid.

15. A method for tuning the isoelectric point of a nanoparticle comprising the step of coating said nanoparticle with a bioderived coating comprising a mixture of chitosan and carboxymethylated chitosan, whereby the isoelectric point is tuned by varying the ratio of chitosan and carboxymethylated chitosan.

16. A method for using theranostic hybrid nanoparticles comprising the steps of:
a. providing optically heatable hybrid nanoparticles having diameters less than about 100 nm and improved contrast functionality with X-ray and CT imaging;
b. delivering said hybrid nanoparticles to a specific tissue;
c. imaging said tissue using at least one of X-ray and CT imaging;
d. optically heating said hybrid nanoparticles located at said specific tissue, whereby optically heating said hybrid nanoparticles results in at least one of:
1. thermal ablation of at least a portion of said specific tissue;
2. release of a diagnostic agent incorporated within said hybrid nanoparticles; and
3. release of a therapeutic agent incorporated within said hybrid nanoparticles.

17. The method of claim 16, wherein each of said hybrid nanoparticles are comprised of a nanoparticle comprising gold and gold sulfide, and a bioderived coating assembled on said nanoparticle.

18. The method of claim 16, wherein a contrast agent is incorporated within said bioderived coating.

19. The method of claim 18, wherein said contrast agent is not released upon optically heating said hybrid nanoparticle.

20. The method of claim 16, wherein said specific tissue is a cancerous tissue.

21. A method for delivering a therapeutic or diagnostic agent to a specific tissue comprising the steps of:
a. providing optically heatable hybrid nanoparticles comprised of:
1. a nanoparticle comprising gold and gold sulfide;
2. a bioderived coating assembled on said nanoparticle;
3. at least one agent from the group consisting of a therapeutic agent and a diagnostic agent, said agent releaseably incorporated within said bioderived coating;
b. delivering said hybrid nanoparticles to a specific tissue;
c. optically heating said hybrid nanoparticles located at said specific tissue, whereby optically heating said hybrid nanoparticles results in release of said at least one agent.
